(19) United States
(12) Patent Application Publication
(10) Pub. No.: US 2010/0160237 A1
(43) Pub. Date: Jun. 24, 2010

(54) DRY FEEDSTUFF FOR CONTROLLING EXCESS WEIGHT AND OBESITY OF DOGS

(75) Inventors: Renaud Sergheraert, Fabregues (FR); Vincent Biourge, Villevielle (FR); Marianne Diez, Bastogne (BE); Tan-Hung Nguyen, St. Ave (FR)

Correspondence Address: JONES DAY, 222 EAST 41ST ST, NEW YORK, NY, 10017, US

(73) Assignee: Royal Canin S.A.

(21) Appl. No.: 12/716,291

(22) Filed: Mar. 3, 2010

Related U.S. Application Data
(63) Continuation of application No. 10/250,977, filed on Apr. 13, 2004.

(30) Foreign Application Priority Data
Jan. 17, 2001 (FR) 0100587

(51) Int. Cl. A61K 38/16; A61P 3/04

(52) U.S. Cl. 514/12

(57) ABSTRACT

The invention concerns a dry feedstuff designed to feed dogs containing at least 35% of proteins relative to dry matter, and/or a protein input more than 110 grams per 1 000 kcal of metabolizable energy, and its use as food, additive or veterinary medicine in particular for controlling excess weight and obesity in dogs.

This application is a continuation of U.S. patent application Ser. No. 10/250,977, filed Apr. 13, 2004, national stage of International Application No. PCT/FR02/00181, filed Jan. 17, 2002, each of which are hereby incorporated by reference in their entireties.

### FIELD OF THE INVENTION

The present invention relates to dry feedstuffs for dogs, which feedstuffs are intended to control their excess weight and their obesity.

### BACKGROUND OF THE INVENTION

The canine species comprises hundreds of breeds whose “standards” are well defined. Thus, the “ideal or optimal” weight of a dog is an important criterion which is well known to breeders and to veterinarians.

However, excess weight and obesity affect from 20 to 30% of dogs in the industrialized countries.

It is considered that a dog is obese when its weight exceeds its ideal weight by 15-20%.

However, simply weighing the animal is insufficient for assessing obesity since it does not give any indication of the animal's body composition. Thus, obesity is defined as being the accumulation of an excessive quantity of adipose tissue in the body. The weight increases when the fatty tissues accumulate. In this way, an excess of adipose tissue in the body and excess weight are closely linked.

When the excess weight reaches or exceeds 20-30%, the animal is at great risk of contracting diseases.

In a veterinary clinic, practitioners have available several methods for determining the composition of the fatty tissue masses and lean tissue masses of the dog, extending from the simplest to the most sophisticated:
scoring body condition: most frequently from 1 to 5, with 5 being the grade for very obese dogs;
morphometric measurements: the percentage of body fat can be estimated using equations whose variables are measurements such as the length of the posterior limbs, the length of the body, the pelvic circumference, the thoracic circumference and the weight;
complex methods performed in specialist laboratories: isotope dilution (using deuterium, for example), bioimpedance, DEXA (dual energy X-ray absorptiometry), etc.

These techniques for assessing obesity and the composition of the adipose and lean tissues are very useful in veterinary medicine since obesity can lead to a large number of problems for the health and life-expectancy of the dogs: diabetes, hypertension, cardiovascular diseases, hypersensitivity to anesthetics, locomotory disturbances, cancers, etc., with this only being a list of the most frequent risks.

Two causes which are at the origin of obesity in dogs and which are frequently blamed are: their sedentary way of life, which results in a lack of physical exercise, and their diet.

In addition to domestic rations which are frequently poorly balanced from the nutritional point of view, dogs are evermore frequently given commercial diets which supply them with all the nutrients recommended by nutritionists, the most well known of whom are those in the National Research Council (N.R.C.) in the United States of America.

Commercial feedstuffs for dogs come in two main categories: dry feedstuffs and wet feedstuffs. While an intermediate category, i.e. semi-wet feedstuffs, also exists, these feedstuffs are not produced in very large amounts. The dry feedstuffs are making the most rapid progress in the market because they are practical to use and easy to preserve. They are also easier to formulate for achieving sought-after nutritional balances. Finally, they are more economical than the wet feedstuffs.

A large number of methods have been proposed for producing dry feedstuffs which are able to control excess weight and obesity in dogs.

Thus, the Association of American Feed Control Officials (AAFCO) has recommended what are termed “light” dry feedstuffs for dogs, having a maximum metabolizable energy of 3 100 kcal/kg, and “lean” feedstuffs, containing a maximum of 10% fats.

Other dry anti-obesity feedstuffs contain high fiber levels.

S. S. Hannah (Proc. 16th American College of Veterinary Internal Medicine Forum, San Diego, Calif., USA, 1998, p. 714, and Proc, Purina Nutrition Forum, Saint Louis, Mo., USA, 1998, pp. 1-5) has shown that the loss of body fat in dogs is greater when 39% of the metabolizable calories are supplied in the form of protein than when the proteins are only supplying 20 or 30% of the calories. The loss of lean tissue is also lower when 39% of the calories are of protein origin than when only 20 or 30% are of protein origin. However, S. S. Hannah did not indicate the protein levels or the metabolizable energy level in the feedstuffs which were tested.

As reported in a recent review by W. J. Burkholder and P. W. Toll (Obesity, in: Small Animal Clinical Nutrition, Hand, Thatcher, Remillard, Roudebush, Ed. Mark Morris Institute, Topeka, Kans., USA, 4^{th} Edition, 2000, pp. 401-430), a very large number of dry feedstuffs have been proposed for achieving loss of weight in obese dogs:

| | | | | | Protein |
|---|---|---|---|---|---|
| Brands and dry feedstuffs | Kcal*/ | Fat | Crude fiber | Protein | (g/1 000 |
| for dogs | kg DM** | (% DM) | (% DM) | (% DM) | kcal)*** |
| Hill's Prescription Diet | 2 966 | 8.4 | 23.5 | 24.8 | 83.6 |
| Canine r/d^{a} | | | | | |
| Hill's Prescription Diet | 3 216 | 8.8 | 16.8 | 16.7 | 51.9 |
| Canine w/d | | | | | |
| Hill's Science Diet Canine | 3 293 | 9.0 | 14.8 | 18.6 | 56.5 |
| Maintenance Light | | | | | |
| Iams Eukanuba Reduced | 4 306 | 10.0 | 4.4 | 21.1 | 48.4 |
| Fat Formula | | | | | |
| Iams Eukanuba Restricted | 4 053 | 6.6 | 1.9 | 19.2 | 47.4 |
| Calorie | | | | | |
| Iams Less Active | 4 281 | 12.5 | 5.6 | 22.2 | 51.9 |
| Leo Specific Fitness CRD | 3 633 | 5.6 | 8.9 | 24.4 | 67.2 |
| Medi-Cal Canine Fibre | 3 078 | | 10.1 | 15.8 | 78.9 |
| Formula | | | | | |
| Medi-Cal Canine Weight | 3 434 | 8.3 | 5.9 | 19.6 | 57.1 |
| Control/Geriatric | | | | | |
| Purina CNM OM-Formula | 2 783 | 6.0 | 15.2 | 22.8 | 81.9 |
| Purina Fit & Trim | 3 100 | 7.4 | 10.8 | 15.3 | 49.3 |
| Purina O.N.E. Reduced | 3 623 | 8.9 | 3.1 | 18.2 | 50.2 |
| Calorie Formula | | | | | |
| Purina Pro Plan Reduced | 3 638 | 9.7 | 2.7 | 15.9 | 43.7 |
| Calorie Formula | | | | | |
| Quaker Cycle Lite | 3 217 | 10.1 | 4.8 | 18.7 | 58.1 |
| Select Care Canine | 3 278 | 10.8 | 14.7 | 25.7 | 78.4 |
| Hifactor Formula | | | | | |
| Waltham/Pedigree Calorie | 3 500 | 8.9 | 1.8 | 32.2 | 92.0 |
| Control/Low Calorie | | | | | |

*value supplied by the manufacturer or recalculated in accordance with N.R.C. 1974 (4 kcal/g of protein and nitrogen-free extract and 9 kcal/g of fat)
**DM = dry matter
***recalculated by the applicant
^{a}feedstuff regarded as being the reference; that which is most frequently prescribed by veterinarians.

As a rule, veterinarians recommend that these anti-obesity feedstuffs be apportioned in a quantity which is sufficient to cause the dogs to lose between 1 and 2% of their weight per week, with greater and more rapid losses in weight running the risk of harming the health of the animals.

Despite so many feedstuffs being proposed for controlling obesity, the incidence of obesity in dogs continues to increase. In addition, these feedstuffs are frequently criticized for causing obese dogs to lose too much lean tissue in addition to losing adipose tissue.

It is therefore necessary to formulate a novel dry feedstuff for dogs which, at one and the same time, meets the criteria of industrial manufacture (preparation using traditional methods, unit constituents which “hold together well”), hypo-caloricity (in order to retain a foodstuff volume which is sufficient for the animal to have a sensation of satiety while nevertheless reducing the supply of energy), completeness and balance from a nutritional point of view (presence of essential fatty acids and of minerals and vitamins), and use in a reducing regime (loss of fatty tissue rather than lean tissue in the animal).

### SUMMARY OF THE INVENTION

The applicant has discovered that it is possible to cause obese dogs to lose weight by offering them a dry feedstuff in which, in particular, the proportion of protein is greater than 35%, preferably 37%, more preferably 39%, of the dry matter (by weight) and in which the protein supplied is greater than 110 grams per 1 000 (thousand) kcal of metabolizable energy, preferably greater than 120 g/1 000 kcal, more preferably greater than 130 g/1 000 kcal.

The metabolizable energy of the feedstuffs, which is a parameter which is well known to the skilled person, is defined as being the difference between the gross energy, on the one hand, and, on the other hand, the energy which is excreted in the feces and the urine. The metabolizable energy can be measured in vivo by placing the dogs in metabolism cages or else calculated from regression equations, the most well known of which are those provided by the N.R.C.

### DETAILED DESCRIPTION OF THE INVENTION

Thus, the use of such a feedstuff according to the invention makes it possible to chiefly reduce the fatty tissues of the animal while maintaining the lean tissues, as the examples demonstrate.

In order to also meet the other criteria which are enumerated above, it is important for the feedstuff according to the invention to possess a hypocalorific character, that is to say have an energy content which is preferably less than 3 100 kcal/kg, in conformity with the recommendations of the AAFCO 2000, as defined in its rules PF9 and PF10.

In order to reduce the energy which is supplied by the feedstuff, it is worthwhile to increase the proportion of fiber in the composition of the feedstuff. Thus, in the feedstuff according to the invention, the proportion of crude fiber is preferably greater than or equal to 10%, more preferably greater than or equal to 14%, based on the total composition of the feedstuff.

In order to be able to offer a dry feedstuff which “holds together well”, it is necessary to add a binder to the feedstuff according to the invention. The binder which is generally used is starch which, however, suffers from the disadvantage of being relatively rich in calories. It is therefore advantageous, in order to maintain the low energy level of the feedstuff according to the invention, to reduce the quantity of binder which is supplied to the feedstuff when it is being manufactured.

Thus, the proportion of starch in the feedstuff according to the invention is preferably less than 20%, more preferably less than or equal to 15% to 12%, and most preferably less than 10%, based on the total composition of the feedstuff.

The feedstuff according to the invention can contain other raw materials, in particular:
at least one source of animal, vegetable, microbial or fungal protein, and
at least one source of slowly or rapidly absorbed carbohydrates, and/or
at least one source of animal or vegetable fat.

In certain embodiments, the feedstuff according to the invention is characterized in that it contains at least 35% protein based on the dry matter.

In certain embodiments, the feedstuff according to the invention is characterized in that the content of protein is at least 110 grams per 1 000 kilocalories of metabolizable energy.

In certain embodiments, the feedstuff according to the invention is characterized in that it contains a content of starch which is less than 20%.

In certain embodiments, the feedstuff according to the invention is characterized in that it contains at least 10% crude fiber.

The feedstuff according to the invention also preferably contains constituents which make it possible to supply the nutrients which are required so as to ensure that the animal is receiving a balanced diet. The feedstuff therefore preferably contains minerals (preferably in an amount greater than 5%) or essential fatty acids, etc.

The invention is also independent of the form in which the dry feedstuff is presented, with it being possible for this form to be an extruded croquette, a flake, granules or a biscuit, with this list not being limiting.

The applicant has discovered that the feedstuff of the invention enables excess weight and/or obesity in dogs to be controlled by making the dogs lose weight and adipose tissue while at the same time not losing too much lean tissue. The feedstuff is also of value as an accompaniment to treatment of disorders generated in dogs by obesity.

Thus, the invention also relates to a feedstuff according to the invention as a veterinary medicament or additive, particularly for treating weight-related disorders in dogs.

### EXAMPLE

The following example, which is not limiting and not exhaustive, illustrates the efficacy of a feedstuff according to the invention.

A dry feedstuff according to the invention for dogs was made up, with this feedstuff being presented in the form of extruded croquettes and having the following analytical composition:
moisture: 6.8%
protein: 39.9% (42.8% of the dry matter)
nitrogen-free extract: 24.3%
fats: 8.4%
crude fiber: 14.0%
starch content: 7.8%
minerals: 6.6%
metabolizable energy:
   energy measured on animals: 2 758 kcal per kilo (2 959 on dry material)
   energy calculated in accordance with NRC85: 2 961 kcal per kilo (3 177 on dry material)
metabolizable energy supplied by protein: 57.9%
quantity of protein/1 000 kcal:
   protein/measured energy: 144.67 grams
   protein/energy calculated in accordance with NRC85: 134.75 grams

This feedstuff was compared with the extruded dry feedstuff Hill's Prescription Diet Canine r/d, which is regarded as being the reference for feedstuffs which are used for slimming dogs, i.e. being the feedstuff which is most frequently prescribed and sold for this purpose. This latter feedstuff has the following analytical characteristics, as listed in the Dictionnaire des Médicaments Vétérinaires et des Produits de SantéAnimale [Dictionary of Veterinary Médicaments and Animal Health Products], Edition du Point Vétérinaire, Maisons-Alfort, France, 1999, pages 1504-1505:
moisture: 9%
protein: 22.3% (24.5% of the dry matter)
nitrogen-free extract: 34.7%
fats: 7.7%
crude fiber: 21.3%
metabolizable energy:
   energy measured on animals: 2 387 kcal per kilo (2 626 on dry material)
   energy calculated in accordance with NRC85: 2 657 kcal per kilo (2 922 on dry material)
metabolizable energy supplied by protein: 33.7%
quantity of protein/1 000 kcal:
   protein/measured energy: 90.49 grams
   protein/energy calculated in accordance with NRC85: 81.29 grams

The experimental feedstuff and the reference feedstuff were apportioned to two groups of obese dogs, with each group comprising two males and two females. Before being introduced into the trial, their actual weights, their ideal weights and their excess weights were the following, as measured and calculated by veterinary specialists:

| | | | Actual | Ideal | Excess |
|---|---|---|---|---|---|
| Feedstuff | Sex | Dog | weight (kg) | weight (kg) | weight (%) |
| Experimental | Male | 1 | 23.45 | 14.7 | 59.5 |
| | Male | 2 | 18.15 | 13.5 | 34.4 |
| | Female | 5 | 20.60 | 14.0 | 47.1 |
| | Female | 6 | 16.70 | 12.0 | 39.2 |
| Hill's r/d | Male | 3 | 19.80 | 14.0 | 41.4 |
| | Male | 4 | 20.90 | 14.2 | 47.2 |
| | Female | 7 | 17.65 | 13.0 | 35.8 |
| | Female | 8 | 18.90 | 14.0 | 35.0 |

The dogs were fed individually with their respective feedstuffs, with the quantities being adjusted in order to ensure a regular weight loss of the order of from 1 to 2% per week, in accordance with the custom for reducing treatments for dogs.

The animals are weighed individually before being introduced into the trial and then regularly at least once per week. In the case of each dog, the trial is stopped when the animal has reached or gone beyond its ideal weight as previously defined.

The body composition of each animal, as regards fats and lean mass, is measured using the deuterium dilution method (reference for the method: H. R. Son, D. A. d'Avignon and D. P. Laflamme, American Journal of Veterinary Research, 1998, 59(5), 529-532) before the animal is introduced into the trial and when it has reached or gone beyond its ideal weight. In this way, it is also possible to measure the composition of the weight which has been lost.

The results at the end of the trial were as follows:

| | | Final | Weight | Composition of the | |
|---|---|---|---|---|---|
| | | weight | loss | weight loss | |
| Feedstuff | Dog | (kg) | (kg) | Fats (%) | Lean mass (%) |
| Experi- | 1 | 13.80 | 9.65 | 72.38 | 27.62 |
| mental | 2 | 12.60 | 5.55 | 81.01 | 18.99 |
| | 5 | 14.35 | 6.25 | 87.50 | 12.50 |
| | 6 | 12.35 | 4.35 | 80.76 | 19.24 |
| | | | Mean: | Mean: | Mean: |
| | | | 6.45 ± 2.27 | 80.41 ± 6.20 | 19.59 ± 6.20 |
| | | | | (114.8%) | (65.4%) |
| Hill's r/d | 3 | 13.00 | 6.80 | 75.58 | 24.42 |
| | 4 | 13.05 | 7.85 | 75.13 | 24.87 |
| | 7 | 12.25 | 5.40 | 63.44 | 36.56 |
| | 8 | 12.30 | 6.60 | 66.07 | 33.93 |
| | | | Mean: | Mean: | Mean: |
| | | | 6.66 ± 1.00 | 70.06 ± 6.22 | 29.94 ± 6.22 |
| | | | | (100%) | (100%) |

These results show that, for equivalent losses in weight, the experimental feedstuff brings about a larger loss of fat tissues and a lower loss of lean tissues, with this being a sought-after favorable effect within the context of a reducing regime.

What is claimed is:

1. A method of reducing more fatty tissues than lean tissues in an obese dog to control excess weight, said method comprising feeding an obese dog in need thereof a dry feedstuff comprising a composition that comprises:
a. a source of animal, vegetable, microbial, or fungal protein;
b. a source of slowly or rapidly absorbed carbohydrates; and
c. a source of animal or vegetable fat,
wherein the protein in the composition is present in an amount of at least 110 grams per 1,000 kcal of metabolizable energy, and wherein the metabolizable energy content of the composition is less than 3,100 kcal/kg.

2. The method of claim 1, wherein the protein in the composition is present in an amount of at least 39 weight percent based on dry matter.

3. The method of claim 1, wherein the protein in the composition is present in an amount of at least 120 grams per 1,000 kcal of metabolizable energy.

4. The method of claim 3, wherein the protein in the composition is present in an amount of at least 130 grams per 1,000 kcal of metabolizable energy.

5. The method of claim 1, wherein the carbohydrate is starch.

6. The method of claim 5, wherein the starch in the composition is present in an amount of less than 20 weight percent based on dry matter.

7. The method of claim 6, wherein the starch in the composition is present in an amount of less than or equal to 15 weight percent based on dry matter.

8. The method of claim 7, wherein the starch in the composition is present in an amount of less than 10 weight percent based on dry matter.

9. The method of claim 1, wherein the composition further comprises a source of crude fiber.

10. The method of claim 9, wherein the crude fiber in the composition is present in an amount of greater than or equal to 10 weight percent based on dry matter.

11. The method of claim 10, wherein the crude fiber in the composition is present in an amount of greater than or equal to 14 weight percent based on dry matter.

12. The method of claim 1, wherein the amount of fatty tissues reduced is about four times the amount of lean tissues reduced.

13. The method of claim 1, wherein the dry feedstuff is a veterinary medicament or an additive.
